(19) United States
(12) Patent Application Publication
(10) Pub. No.: US 2025/0186790 A1
(43) Pub. Date: Jun. 12, 2025

(54) Implantable Electrical Connector Assembly

(71) Applicant: TC1 LLC, St. Paul, MN (US)

(72) Inventors: Daniel I. Harjes, Carlisle, MA (US); Jeff Iudice, Lowell, MA (US); Soy Truong, Tigard, OR (US); Chanthy Luy, Tigard, OR (US); Jason Elledge, Tigard, OR (US); Joseph P. Sylvester, JR., Andover, MA (US); Michael Morado, Escalon, CA (US); Fabian Frigon Franco, Livermore, CA (US); Lindsay Clough, Portland, OR (US); John Hai Nguyen, San Ramon, CA (US)

(73) Assignee: TC1 LLC, St. Paul, MN (US)

(21) Appl. No.: 19/059,628

(22) Filed: Feb. 21, 2025

Related U.S. Application Data
(63) Continuation of application No. PCT/US2023/073022, filed on Aug. 28, 2023.
(60) Provisional application No. 63/401,831, filed on Aug. 29, 2022.

(51) Int. Cl. A61N 1/375; H01R 24/58

(52) U.S. Cl. CPC A61N 1/3752; H01R 24/58; H01R 2201/12

(57) ABSTRACT

The disclosure is directed to implantable electrical connector assemblies and methods of fabrication of the implantable electrical connector assemblies. An implantable electrical connector includes a male electrical connector and a female electrical connector. The male electrical connector includes annular electrical contacts mounted to and spaced apart along an elongated support member. The female electrical connector includes female contact assemblies. Each of the female contact assemblies includes a circular coil spring disposed within an annular conductive member. The circular coil spring is retained within the annular conductive member between a first-side non-conductive retention member and the second-side non-conductive retention member.

### CROSS REFERENCE TO RELATED APPLICATIONS

The present application is a Continuation of PCT/US2023/073022 filed Aug. 28, 2023; which claims the benefit of U.S. Provisional Appln. No. 63/401,831 filed Aug. 29, 2022, the disclosures which are incorporated herein by reference in their entirety for all purposes.

### FIELD OF DISCLOSURE

This disclosure relates generally to implantable medical devices and related components. More particularly, this disclosure relates to an implantable electrical connector assembly for the implantable medical device.

### BACKGROUND

Implantable devices employ an electrical connector to communicate signals between a controller, a patient's organ (e.g., heart) or a nerve, and/or other implanted devices. The implantable devices may include one, two, or more electrical connectors. For example, Ventricular assist devices, known as VADs, use two electrical connectors. VADs are implantable blood pumps used for both short-term (i.e., days, months) and long-term applications (i.e., years or a lifetime) where a patient's heart is incapable of providing adequate circulation, commonly referred to as heart failure or congestive heart failure. VAD employs two electrically isolated electrical connectors-one coupled to the blood pump and another to an energy transfer system.

According to the American Heart Association, more than five million Americans are living with heart failure, with about 670,000 new cases diagnosed every year. People with heart failure often have shortness of breath and fatigue. Years of living with blocked arteries or high blood pressure can leave a heart too weak to pump enough blood to the body. As symptoms worsen, advanced heart failure develops. A patient may use a VAD while awaiting a heart transplant or as a long-term destination therapy. In another example, a patient may use a VAD while their own native heart recovers. Thus, a VAD can supplement a weak heart (i.e., partial support) or can effectively replace the natural heart's function. VAD systems of the present invention can be fully implanted in the patient's body and powered by an implantable electrical power source inside the patient's body.

### BRIEF SUMMARY

The present disclosure relates to implantable medical devices including an implantable electrical connector. The implantable electrical connectors include use a female electrical connector with a conductive member coupled to a full circumferential circular springs and disposed within a connector body. Existing female electrical connector are machined, which is expensive and cost or process prohibitive, e.g., especially when machined out of an expensive corrosion resistant metal like platinum. The present disclosure provides an electrical connector having a rigid, robust, reliable, and functional composite female contact assembly which can significantly reduce the cost of the female electrical connector from stock platinum. For example, the female electrical connector includes an annular conductive member having simple geometric construction such that it can be extruded without loss of precious metal and further mounted on non-conductive side members. A circular spring is disposed at the center of the annular conductive member and retained between the non-conductive side members. The non-conductive side members can be made of polyetheretherketone (PEEK) and the conductive member is made of platinum iridium. It can advantageously eliminate loss of precious metal by extruding a part rather than machining and generating wasteful platinum chips. It also allows for modular scalability of an implantable header of the medical device with minimal disruption in cost of goods.

Thus, in one aspect, an implantable electrical connector assembly including a female electrical connector is described. The implantable electrical connector assembly includes a male electrical connector and a female electrical connector. The male electrical connector includes an elongated electrical contact support member and annular electrical contacts mounted to and spaced apart along the elongated electrical contact support member. The female electrical connector includes a connector body having an elongated receptacle and female contact assemblies disposed in and distributed along the elongated receptacle for interfacing with the annular electrical contacts of the male electrical connector. Each of the female contact assemblies comprises a circular coil spring, an annular conductive member, a first-side non-conductive retention member, and a second-side non-conductive retention member. The circular coil spring is disposed within the annular conductive member. The first-side non-conductive retention member is mounted to the annular conductive member and disposed on a first side of the circular coil spring. The second-side non-conductive retention member is mounted to the annular conductive member and disposed on a second side of the circular coil spring opposite to the first side of the circular coil spring. The circular coil spring is retained between and by the first-side non-conductive retention member and the second-side non-conductive retention member. In many embodiments, the annular conductive member has a cylindrical shape. The annular conductive member is formed from a cylindrical extrusion.

In many embodiments, the annular conductive member includes a cylindrical inner surface, an annular first end surface, and an annular second end surface. A cylindrical outer surface of the first-side non-conductive retention member is interfaced with a first end portion of the cylindrical inner surface. Each of the non-conductive retention members include an annular flange portion. The annular flange portion of the first-side non-conductive retention member is interfaced with the annular first end surface of the annular conductive member. A cylindrical outer surface of the second-side non-conductive retention member is interfaced with a second end portion of the cylindrical inner surface. The annular flange of the second-side non-conductive retention member is interfaced with the annular second end surface the annular conductive member. The circular coil spring is configured to be interfaced with a central portion of the annular conductive member that extends between the first end portion and the second end portion of the cylindrical inner surface to electrically couple the annular conductive member with a respective one of the annular electrical contacts of the male electrical connector. In many embodiments, the female electrical connector further includes wiper seal assemblies. Each of the female contact assemblies is disposed between two of the wiper seal assemblies. Each of the wiper seal assemblies comprises an annular housing and an annular seal supported by the annular housing. The annular seal is configured to sealing engage the male electrical connector, and wiper seal assembly is configured to cooperate with the male electrical connector to block passage of fluid past the annular seal and along the male electrical connector.

In some embodiments, the annular conductive member includes a cylindrical outer surface, an annular first end surface, and an annular second end surface. A cylindrical inner surface of the first-side non-conductive retention member is interfaced with a first end portion of the cylindrical outer surface. An annular flange of the first-side non-conductive retention member is interfaced with the annular first end surface. A cylindrical inner surface of the second-side non-conductive retention member is interfaced with a second end portion of the cylindrical outer surface. An annular flange of the second-side non-conductive retention member is interfaced with the annular second end surface. The circular coil spring is configured to be interfaced with a central portion of the annular conductive member that extends between the first end portion and the second end portion of the cylindrical inner surface to electrically couple the annular conductive member with a respective one of the annular electrical contacts of the male electrical connector. The female electrical connector further comprises wiper seal assemblies. Each of the female contact assemblies is disposed between two of the wiper seal assemblies, and each of the wiper seal assemblies includes an annular housing and an annular seal supported by the annular housing. The annular seal is configured to sealing engage the male electrical connector, and the wiper seal assembly is configured to cooperate with the male electrical connector to block passage of fluid past the annular seal and along the male electrical connector.

In many embodiments, the annular conductive member has an internal diameter greater than 3.2 mm. Each of the first-side non-conductive retention member and the second-side non-conductive retention member is made of polyetheretherketone (PEEK) or thermoplastic polyurethanes (TPU). The annular conductive member is made of a platinum-iridium alloy.

According to another aspect, a method of fabricating a female electrical connector of an implantable electrical connector assembly is described. The method includes receiving a connector body comprising an elongated receptacle, receiving female contact assemblies, and retaining the female contact assemblies within the elongated receptacle of the connector body. Each of the female contact assemblies includes a cylindrical conductive member, a circular coil spring, a first-side non-conductive retention member, and a second-side non-conductive retention member, wherein the circular coil spring is disposed within the cylindrical conductive member. The first-side non-conductive retention member is mounted to the cylindrical conductive member and disposed on a first side of the circular coil spring. The second-side non-conductive retention member is mounted to the cylindrical conductive member and disposed on a second side of the circular coil spring opposite to the first side of the circular coil spring. The circular coil spring is retained between and by the first-side non-conductive retention member and the second-side non-conductive retention member.

In many embodiments, the method further includes retaining wiper seal assemblies within elongated receptacle of the connector body. Each of the female contact assemblies is disposed between two of the wiper seal assemblies. Each of the wiper seal assemblies comprises an annular housing and an annular seal supported by the annular housing and configured to sealingly engage a male electrical connector, and each of the wiper seal assemblies is configured to cooperate with the male electrical connector to block passage of fluid.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and can or cannot represent actual or preferred values or dimensions.

FIG. 1 illustrates a medical system that includes a fully implantable transcutaneous energy transmission system receiver, an implantable controller, and a ventricular assist device (VAD), in accordance with many embodiments.

FIG. 2 is an illustration of a blood circulatory support system that includes the VAD implanted in a patient's body, in accordance with many embodiments.

FIG. 3 is a partially exploded view of implanted components of VAD Blood Pump the implanted circulatory support system of FIG. 2.

FIG. 4 is an illustration of the VAD of FIG. 1 attached to the patient's heart to augment blood pumping by the patient's left ventricle.

FIG. 5 is a cross-sectional view of the VAD of FIG. 4.

FIG. 6 is a schematic diagram of an embodiment of the implanted controller of FIG. 1.

FIG. 7 is a female contact assembly of a female electrical connector, according to many embodiments.

FIG. 8 is an exploded view of the female contact assembly of FIG. 7.

FIG. 9A and FIG. 9B are cross-section views of the female contact assembly of FIG. 7.

FIG. 10 is an assembled or un-exploded view showing the female contact assembly of FIG. 7 and an adjacent wiper seal.

FIG. 11 is a female electrical connector including the female contact assembly of FIG. 7, according to many embodiments.

FIG. 12 is the female electrical connector of FIG. 11 illustrating a coupling between the female contact assembly of FIG. 7 and a wiper seal.

FIG. 13 illustrates different views of the female electrical connector of FIG. 11 including (A) a cross-section view, (B) a side view, and (C) an enlarged view of a portion of the female electrical connector.

FIG. 14 is an electrical connector including the female electrical connector of FIG. 11 and a male electrical connector.

FIG. 15 is a cross-section view the female electrical connector of FIG. 11 and the male electrical connector of FIG. 14.

FIG. 16 is a cross-section view of the electrical connector showing the female electrical connector of FIG. 11 coupled to the male electrical connector of FIG. 14.

FIG. 17 is a method of fabricating an implantable female electrical connector assembly for an implantable medical device, according to many embodiments.

### DETAILED DESCRIPTION OF THE DISCLOSURE

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s). However, it will be apparent to those skilled in the art that the disclosed embodiment(s) can be practiced without those specific details. In some instances, well-known structures and components can be shown in block diagram form in order to avoid obscuring the concepts of the disclosed subject matter. In the drawings, like reference numerals represent like parts throughout the several views.

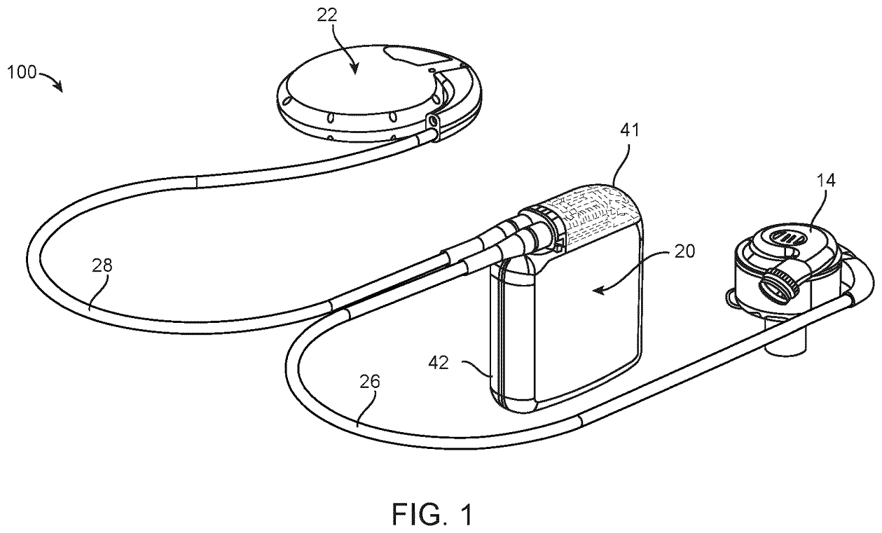

FIG. 1 illustrates a medical system 100 including an implantable medical device 20 couplable to one or more components of the medical system used for treating a medical condition of a patient. The implantable medical device 20 can be an implanted controller and battery configured to control and/or provide power to the one or more components of the medical system. Accordingly, the implantable medical device 20 may also be referred to as an implantable controller and battery 20. The implantable controller and battery 20 includes a housing 42 and a header 41 coupled to the housing 42. In many embodiments, the housing 42 can be made of titanium or like biocompatible materials and the header 41 can be made of a resin (e.g., epoxy or other polymers) or plastic by molding it on the housing 42. The housing 42 is hermetically sealed.

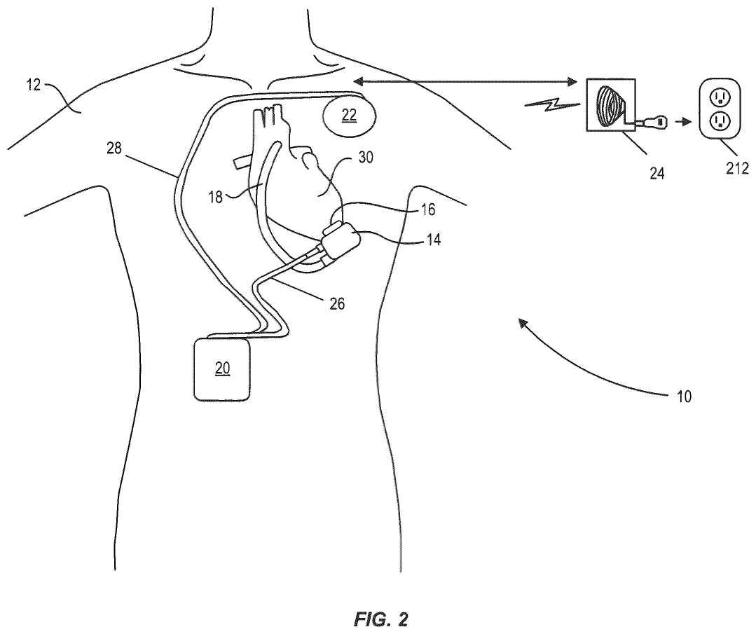

FIG. 2 illustrates a blood circulation assist system 10 using the implantable controller and battery 20 in conjunction with a ventricular assist device (VAD) 14 implanted in a patient 12. In many embodiments described herein, the blood circulatory assist system 10 includes the implantable VAD 14 coupled to a patient's heart 30, the implantable controller and battery 20 that controls operation of the VAD 14 and powers the VAD 14, and an implantable transcutaneous energy transfer system (TETS) power receiver 22 that supplies power to the implantable controller and battery 20. The VAD 14 as illustrated can be a centrifugal flow blood pump, or an axial flow blood pump, that can be operated in a pulsatile mode that produces a blood pressure pulse via an increase in the rotational speed of the VAD or in a constant speed mode in which the VAD rotational rate is held constant.

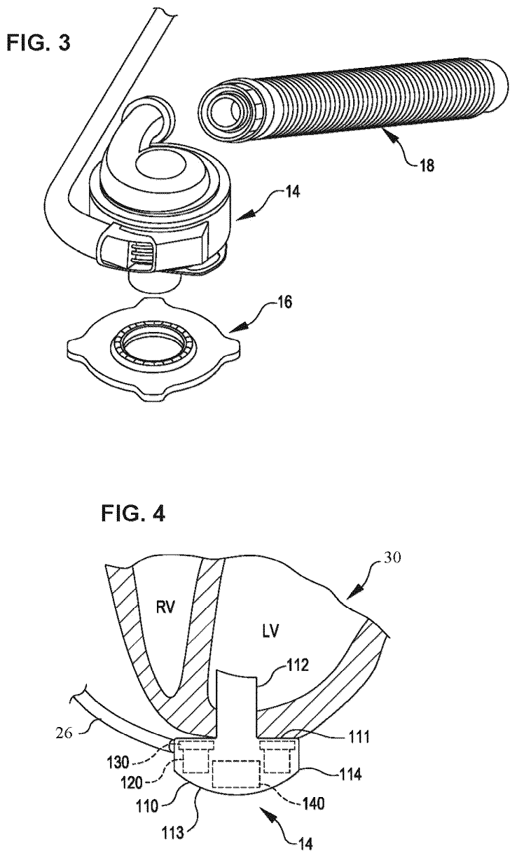

Referring to FIGS. 1 through 3, the blood circulation assist system 10 further includes an implantable ventricular cuff 16, an implantable outflow cannula 18, an external TETS power transmitter 24, an implantable controller-to-VAD connection cable 26, and an implantable TETS receiver-to-controller connection cable 28. The VAD 14 can be coupled to an apex of the left ventricle via the ventricular cuff 16, as illustrated, or the right ventricle, or a separate VAD can be attached to each of the ventricles of the heart 30. Accordingly, depending on the medical application, the system 10 can also be referred as a fully implantable left ventricular assist system (FILVAS) 10. Examples of such FILVAS systems are further described in U.S. Pat. Nos. 8,562,508 and 9,079,043, all of which are incorporated herein by reference for all purposes in their entirety.

The VAD 14, as described in more detail below, can be capable of pumping the entire flow of blood delivered to the left ventricle from the pulmonary circulation (i.e., up to 10 liters per minute). Related blood pumps applicable to the present disclosure are also described in in U.S. Pat. Nos. 5,695,471, 6,071,093, 6,116,862, 6,186,665, 6,234,772, 6,264,635, 6,688,861, 7,699,586, 7,976,271, 7,997,854, 8,007,254, 8,152,493, 8,419,609, 8,652,024, 8,668,473, 8,852,072, 8,864,643, 8,882,744, 9,068,572, 9,091,271, 9,265,870, and 9,382,908, all of which are incorporated herein by reference for all purposes in their entirety. The VAD 14 can be attached to the heart 30 via the ventricular cuff 16, which can be sewn to the heart 30 and coupled to the VAD 14. In the illustrated embodiment, the output of the VAD 14 connects to the ascending aorta via the outflow cannula 18 so that the VAD 14 effectively diverts blood from the left ventricle and propels it to the aorta for circulation through the rest of the patient's vascular system.

The controller-to-VAD connection cable 26 connects the VAD 14 to the implantable controller and battery 20, which monitors the system 10 operation. Related controller systems applicable to the present disclosure are described in greater detail below and in U.S. Pat. Nos. 5,888,242, 6,991,595, 8,323,174, 8,449,444, 8,506,471, 8,597,350, and 8,657,733, EP 1812094, and U.S. Patent Publication Nos. 2005/0071001 and 2013/0314047, all of which are incorporated herein by reference for all purposes in their entirety.

The implantable controller and battery 20 is configured to supply power to and control operation of the VAD 14. The implantable controller and battery 20 is configured to be implanted within the patient 12 in a suitable location spaced apart from the VAD 14 and operatively coupled with the VAD 14 via the controller-to-VAD connection cable 26.

The TETS power receiver 22 is configured to wirelessly receive power transmitted by the external TETS power transmitter 24, which is outside the body for powering operation of the system 10. The TETS power receiver 22 is configured to be implanted within the patient 12 in a suitable location spaced apart from the VAD 14 and the controller and battery 20. The TETS power receiver 22 is operatively coupled with and supplies power to the controller and battery 20 via the implantable TETS power receiver-to-controller connection cable 28. Further, the TETS power transmitter 24 is configured to be coupled to an electric power source 212 such as an electrical wall outlet or other suitable external power sources.

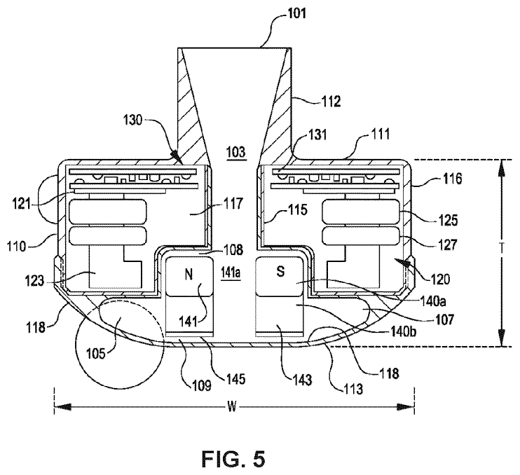

With reference to FIG. 4 and FIG. 5, the VAD 14 has a circular or puck-shaped housing 110 and is shown implanted within the patient 12 with a first face 111 of the housing 110 positioned against the patient's heart 30 and a second face 113 of the housing 110 facing away from the heart 30. The first face 111 of the housing 110 includes an inlet cannula 112 extending into the left ventricle LV of the heart 30. The second face 113 of the housing 110 has a chamfered edge 114 to avoid irritating other tissue that may come into contact with the VAD 14, such as the patient's diaphragm. To construct the illustrated shape of the puck-shaped housing 110 in a compact form, a stator 120 and electronics 130 of the VAD 14 are positioned on the inflow side of the housing toward first face 111, and a rotor 140 of the VAD 14 is positioned along the second face 113. This positioning of the stator 120, electronics 130, and rotor 140 permits the edge 114 to be chamfered along the contour of the rotor 140, as illustrated in at least FIG. 4 and FIG. 5, for example.

Referring to FIG. 5, the VAD 14 includes a dividing wall 115 within the housing 110 defining a blood flow conduit 103. The blood flow conduit 103 extends from an inlet opening 101 of the inlet cannula 112 through the stator 120 to an outlet opening 105 defined by the housing 110. The rotor 140 is positioned within the blood flow conduit 103. The stator 120 is disposed circumferentially about a first portion 140a of the rotor 140, for example about a permanent magnet 141. The stator 120 is also positioned relative to the rotor 140 such that, in use, blood flows within the blood flow conduit 103 through the stator 120 before reaching the rotor 140. The permanent magnet 141 has a permanent magnetic north pole N and a permanent magnetic south pole S for combined active and passive magnetic levitation of the rotor 140 and for rotation of the rotor 140. The rotor 140 also has a second portion 140b that includes impeller blades 143. The impeller blades 143 are located within a volute 107 of the blood flow conduit such that the impeller blades 143 are located proximate to the second face 113 of the housing 110.

The puck-shaped housing 110 further includes a peripheral wall 116 that extends between the first face 111 and a removable cap 118. As illustrated, the peripheral wall 116 is formed as a hollow circular cylinder having a width W between opposing portions of the peripheral wall 116. The housing 110 also has a thickness T between the first face 111 and the second face 113 that is less than the width W. The thickness T is from about 0.5 inches to about 1.5 inches, and the width W is from about 1 inch to about 4 inches. For example, the width W can be approximately 2 inches, and the thickness T can be approximately 1 inch.

The peripheral wall 116 encloses an internal compartment 117 that surrounds the dividing wall 115 and the blood flow conduit 103, with the stator 120 and the electronics 130 disposed in the internal compartment 117 about the dividing wall 115. The removable cap 118 includes the second face 113, the chamfered edge 114, and defines the outlet opening 105. The cap 118 can be threadedly engaged with the peripheral wall 116 to seal the cap 118 in engagement with the peripheral wall 116. The cap 118 includes an inner surface 118a of the cap 118 that defines the volute 107 that is in fluid communication with the outlet opening 105.

Within the internal compartment 117, the electronics 130 are positioned adjacent to the first face 111 and the stator 120 is positioned adjacent to the electronics 130 on an opposite side of the electronics 130 from the first face 111. The electronics 130 include circuit boards 131 and various components carried on the circuit boards 131 to control the operation of the VAD 14 (e.g., magnetic levitation and/or drive of the rotor) by controlling the electrical supply to the stator 120. The housing 110 is configured to receive the circuit boards 131 within the internal compartment 117 generally parallel to the first face 111 for efficient use of the space within the internal compartment 117. The circuit boards also extend radially inward towards the dividing wall 115 and radially-outward towards the peripheral wall 116. For example, the internal compartment 117 is generally sized no larger than necessary to accommodate the circuit boards 131, and space for heat dissipation, material expansion, potting materials, and/or other elements used in installing the circuit boards 131. Thus, the external shape of the housing 110 proximate the first face 111 generally fits the shape of the circuits boards 131 closely to provide external dimensions that are not much greater than the dimensions of the circuit boards 131.

With continued reference to FIG. 5, the stator 120 includes a back iron 121 and pole pieces 123a-123f arranged at intervals around the dividing wall 115. The back iron 121 extends around the dividing wall 115 and is formed as a generally flat disc of a ferromagnetic material, such as steel, in order to conduct magnetic flux. The back iron 121 is arranged beside the control electronics 130 and provides a base for the pole pieces 123a-123f.

Each of the pole piece 123a-123f is L-shaped and has a drive coil 125 for generating an electromagnetic field to rotate the rotor 140. For example, the pole piece 123a has a first leg 124a that contacts the back iron 121 and extends from the back iron 121 towards the second face 113. The pole piece 123a can also have a second leg 124b that extends from the first leg 124a through an opening of a circuit board 131 towards the dividing wall 115 proximate the location of the permanent magnet 141 of the rotor 140. In an aspect, each of the second legs 124b of the pole pieces 123a-123f is sticking through an opening of the circuit board 131. In an aspect, each of the first legs 124a of the pole pieces 123a-123f is sticking through an opening of the circuit board 131. In an aspect, the openings of the circuit board are enclosing the first legs 124a of the pole pieces 123a-123f.

In a general aspect, the VAD 14 can include one or more Hall sensors that may provide an output voltage, which is directly proportional to a strength of a magnetic field that is located in between at least one of the pole pieces 123a-123f and the permanent magnet 141, and the output voltage may provide feedback to the control electronics 130 of the VAD 14 to determine if the rotor 140 and/or the permanent magnet 141 is not at its intended position for the operation of the VAD 14. For example, a position of the rotor 140 and/or the permanent magnet 141 can be adjusted, e.g., the rotor 140 or the permanent magnet 141 may be pushed or pulled towards a center of the blood flow conduit 103 or towards a center of the stator 120.

Each of the pole pieces 123a-123f also has a levitation coil 127 for generating an electromagnetic field to control the radial position of the rotor 140. Each of the drive coils 125 and the levitation coils 127 includes multiple windings of a conductor around the pole pieces 123a-123f. Particularly, each of the drive coils 125 is wound around two adjacent ones of the pole pieces 123, such as pole pieces 123d and 123e, and each levitation coil 127 is wound around a single pole piece. The drive coils 125 and the levitation coils 127 are wound around the first legs of the pole pieces 123, and magnetic flux generated by passing electrical current though the coils 125 and 127 during use is conducted through the first legs and the second legs of the pole pieces 123 and the back iron 121. The drive coils 125 and the levitation coils 127 of the stator 120 are arranged in opposing pairs and are controlled to drive the rotor and to radially levitate the rotor 140 by generating electromagnetic fields that interact with the permanent magnetic poles S and N of the permanent magnet 141. Because the stator 120 includes both the drive coils 125 and the levitation coils 127, only a single stator is needed to levitate the rotor 140 using only passive and active magnetic forces. The permanent magnet 141 in this configuration has only one magnetic moment and is formed from a monolithic permanent magnetic body 141. For example, the stator 120 can be controlled as discussed in U.S. Pat. No. 6,351,048, the entire contents of which are incorporated herein by reference for all purposes. The control electronics 130 and the stator 120 receive electrical power, data, and control signals from the implanted controller and battery 20 via the controller-to-VAD connection cable 26 (FIG. 4). Further related patents, namely U.S. Pat. Nos. 5,708,346, 6,053,705, 6,100,618, 6,222,290, 6,249,067, 6,278,251, 6,351,048, 6,355,998, 6,634,224, 6,879,074, and 7,112,903, are incorporated herein by reference for all purposes in their entirety.

The rotor 140 is arranged within the housing 110 such that its permanent magnet 141 is located upstream of impeller blades in a location closer to the inlet opening 101. The permanent magnet 141 is received within the blood flow conduit 103 proximate the second legs 124b of the pole pieces 123 to provide the passive axial centering force though interaction of the permanent magnet 141 and ferromagnetic material of the pole pieces 123. The permanent magnet 141 of the rotor 140 and the dividing wall 115 form a gap 108 between the permanent magnet 141 and the dividing wall 115 when the rotor 140 is centered within the dividing wall 115. The gap 108 may be from about 0.2 millimeters to about 2 millimeters. For example, the gap 108 can be approximately 1 millimeter. The north permanent magnetic pole N and the south permanent magnetic pole S of the permanent magnet 141 provide a permanent magnetic attractive force between the rotor 140 and the stator 120 that acts as a passive axial centering force that tends to maintain the rotor 140 generally centered within the stator 120 and tends to resist the rotor 140 from moving towards the first face 111 or towards the second face 113. When the gap 108 is smaller, the magnetic attractive force between the permanent magnet 141 and the stator 120 is greater, and the gap 108 is sized to allow the permanent magnet 141 to provide the passive magnetic axial centering force having a magnitude that is adequate to limit the rotor 140 from contacting the dividing wall 115 or the inner surface 118a of the cap 118. The rotor 140 also includes a shroud 145 that covers the ends of the impeller blades 143 facing the second face 113 that assists in directing blood flow into the volute 107. The shroud 145 and the inner surface 118a of the cap 118 form a gap 109 between the shroud 145 and the inner surface 118a when the rotor 140 is levitated by the stator 120. The gap 109 is from about 0.2 millimeters to about 2 millimeters. For example, the gap 109 is approximately 1 millimeter.

As blood flows through the blood flow conduit 103, blood flows through a central aperture 141a formed through the permanent magnet 141. Blood also flows through the gap 108 between the rotor 140 and the dividing wall 115 and through the gap 109 between the shroud 145 and the inner surface 108a of the cap 118. The gaps 108 and 109 are large enough to allow adequate blood flow to limit clot formation that may occur if the blood is allowed to become stagnant. The gaps 108 and 109 are also large enough to limit pressure forces on the blood cells such that the blood is not damaged when flowing through the VAD 14. As a result of the size of the gaps 108 and 109 limiting pressure forces on the blood cells, the gaps 108 and 109 are too large to provide a meaningful hydrodynamic suspension effect. That is to say, the blood does not act as a bearing within the gaps 108 and 109, and the rotor is only magnetically levitated. In various embodiments, the gaps 108 and 109 are sized and dimensioned so the blood flowing through the gaps forms a film that provides a hydrodynamic suspension effect. In this manner, the rotor can be suspended by magnetic forces, hydrodynamic forces, or both.

Because the rotor 140 is radially suspended by active control of the levitation coils 127 as discussed above, and because the rotor 140 is axially suspended by passive interaction of the permanent magnet 141 and the stator 120, no magnetic field generating rotor levitation components are needed proximate the second face 113. The incorporation of all the components for rotor levitation in the stator 120 (i.e., the levitation coils 127 and the pole pieces 123) allows the cap 118 to be contoured to the shape of the impeller blades 143 and the volute 107. Additionally, incorporation of all the rotor levitation components in the stator 120 eliminates the need for electrical connectors extending from the compartment 117 to the cap 118, which allows the cap to be easily installed and/or removed and eliminates potential sources of pump failure.

In use, the drive coils 125 of the stator 120 generates electromagnetic fields through the pole pieces 123 that selectively attract and repel the magnetic north pole N and the magnetic south pole S of the rotor 140 to cause the rotor 140 to rotate within stator 120. For example, the one or more Hall sensors may sense a current position of the rotor 140 and/or the permanent magnet 141, wherein the output voltage of the one or more Hall sensors may be used to selectively attract and repel the magnetic north pole N and the magnetic south pole S of the rotor 140 to cause the rotor 140 to rotate within stator 120. As the rotor 140 rotates, the impeller blades 143 force blood into the volute 107 such that blood is forced out of the outlet opening 105. Additionally, the rotor draws blood into VAD 14 through the inlet opening 101. As blood is drawn into the blood pump by rotation of the impeller blades 143 of the rotor 140, the blood flows through the inlet opening 101 and flows through the control electronics 130 and the stator 120 toward the rotor 140. Blood flows through the aperture 141a of the permanent magnet 141 and between the impeller blades 143, the shroud 145, and the permanent magnet 141, and into the volute 107. Blood also flows around the rotor 140, through the gap 108 and through the gap 109 between the shroud 145 and the inner surface 118a of the cap 118. The blood exits the volute 107 through the outlet opening 105, which may be coupled to the outflow cannula 18.

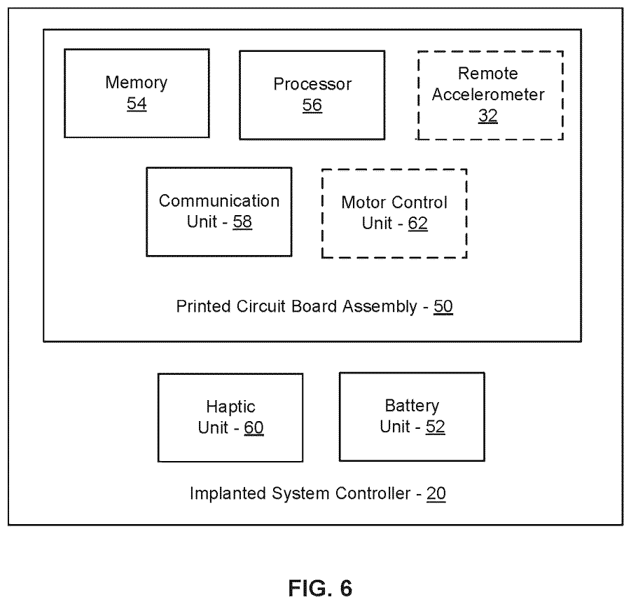

FIG. 6 is a schematic diagram of the embodiment of the implantable controller and battery 20 of FIG. 1. The implantable controller and battery 20 includes a printed circuit board assembly (PCBA) 50, a controller battery unit 52, and a haptic unit 60. In the illustrated embodiment, the PCBA 50 includes a memory 54, a processor 56, and a communication unit 58. In some embodiments, the PCBA 50 includes a motor control unit 62 configured to control drive currents supplied to the motor stator 120 of the VAD 14 to control the rotational rate of the rotor/impeller 140 to control the flow rate of the blood flow through the VAD 14. In some embodiments, PCBA 50 can optionally include a remote accelerometer 32 to measure accelerations induced via operation of the VAD 14. The remote accelerometer 32 can alternately be attached to the inner surface of the housing 42 and operatively coupled with another PCBA.

The memory 54 can store suitable instructions executable by the processor 56 for processing, for example, automatically adjust VAD impeller rotational speed in response to the physiologic demand of the patient, determine any suitable number of physiologic states of the patient, monitor conditions of the VAD 14, and/or other functionalities associated with other components such as the remote accelerometer. The controller battery unit 52 can store energy used to operate the VAD 14, the controller and battery 20, and/or the TETS power receiver 22 during time periods when power is not being received by the TETS power receiver coil. The communication unit 58 can be configured to communicate control commands to the VAD 14 over the implantable controller-to-VAD connection cable 26. The communication unit 58 can also include a suitable wireless communication unit for receiving programming updates and/or for transmitting alarms, VAD operational data, and/or patient physiologic data to an external system monitor.

The controller and battery 20 can be configured so that the haptic unit 60 is operated to generate a haptic alarm to alert the patient that power stored in the controller battery unit 52 and/or the TETS receiver battery unit 40 has dropped below a suitable minimum threshold so that the patient can take action to use the TETS power transmitter 24 to transmit power to the TETS power receiver 22 to recharge the controller battery unit 52 and/or the TETS power receiver battery unit. To guard against a prolonged latent failure of the haptic unit 60, the controller and battery 20 can periodically command operation of the haptic unit 60 to determine whether the haptic unit 60 operated properly or is in a failed state. If the controller and battery 20 determines that the haptic unit 60 is in a failed state, the controller and battery 20 can communicate a suitable alarm indicating the failure of the haptic unit 60 via wireless communication by the communication unit 58.

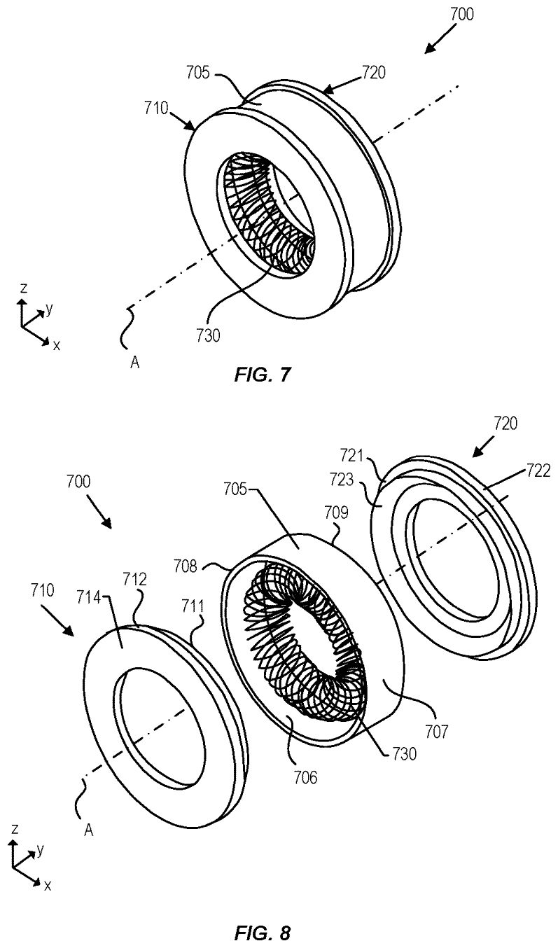

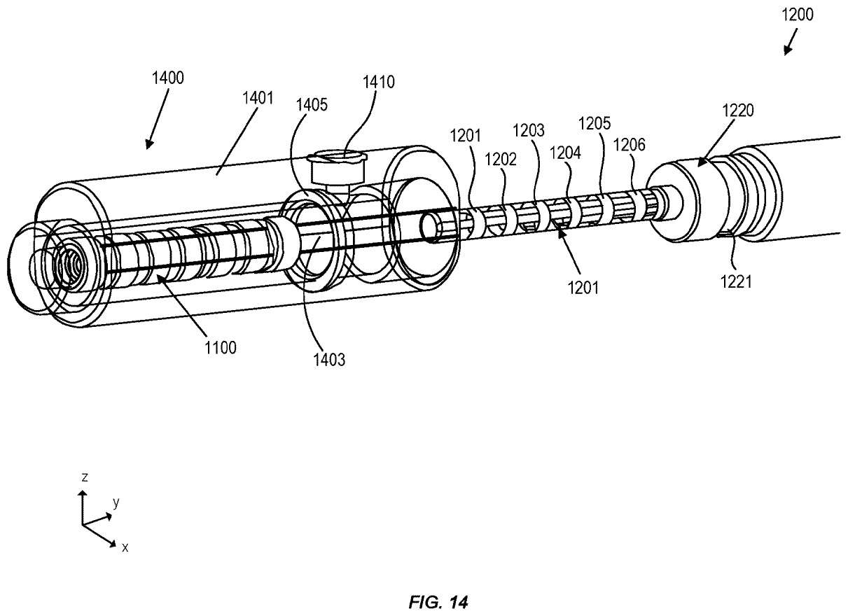

FIG. 7 through FIG. 13 illustrate components and assembly of a female electrical connector. FIG. 14 through 16 illustrate an implantable electrical connector comprising the female electrical connector and a male electrical connector. The implantable electrical connector assembly can be used in various applications including, but not limited to, different implantable medical devices such as VAD, neurostimulators. For example, as shown in FIG. 1, the implantable medical device 20 includes a header 41 configured to include two female electrical connector (e.g., 1400 of FIG. 14) spaced apart and electrically isolated from each other. Each of these female electrical connectors are configured to receive a male electrical connector. For example, as shown in FIG. 1, a first female electrical connector is configured to facilitate power supply and communication between the VAD 14 and the controller and battery 20 via the implantable controller-to-VAD connection cable 26. A second female electrical connector is configured to facilitate power and communication between the TETS power receiver 22 and the controller and battery 20 via the implantable TETS power receiver-to-controller connection cable 28.

FIG. 7 through FIG. 9 illustrate a female contact assembly 700 that can be used to build a female electrical connector, according to many embodiments. The female contact assembly 700 includes an annular conductive member 705, a first-side non-conductive retention member 710, and a second-side non-conductive retention member 720, and a circular coil spring 730. The annular conductive member 705 includes an inner surface 706, an outer surface 707, an annular first end surface 708, and an annular second end surface 709. The first-side non-conductive retention member 710 is mounted to the annular conductive member 705 at a first end of the annular conductive member 705. The second-side non-conductive retention member 720 is mounted to the annular conductive member 705 at a second end of the annular conductive member 705. The circular coil spring 730 is disposed within the annular conductive member 705. These components 705, 710, 720, and 730 of the female contact assembly 700 are coaxially aligned along a central axis A.

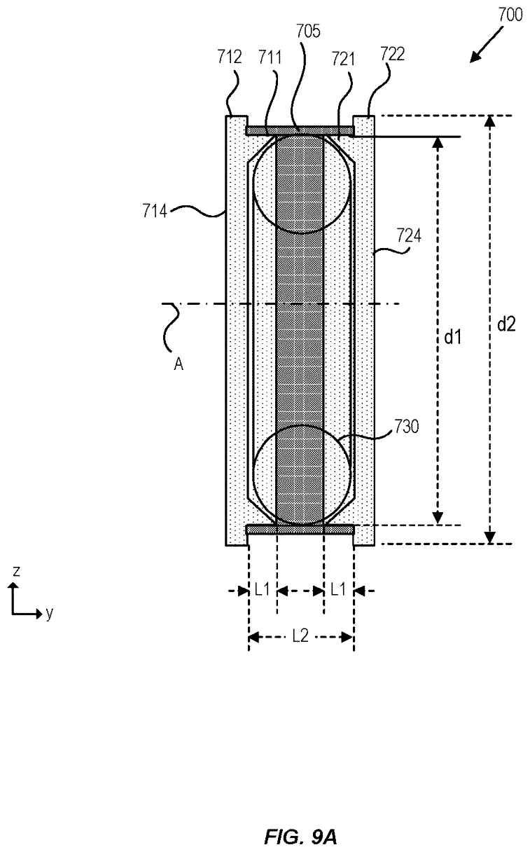

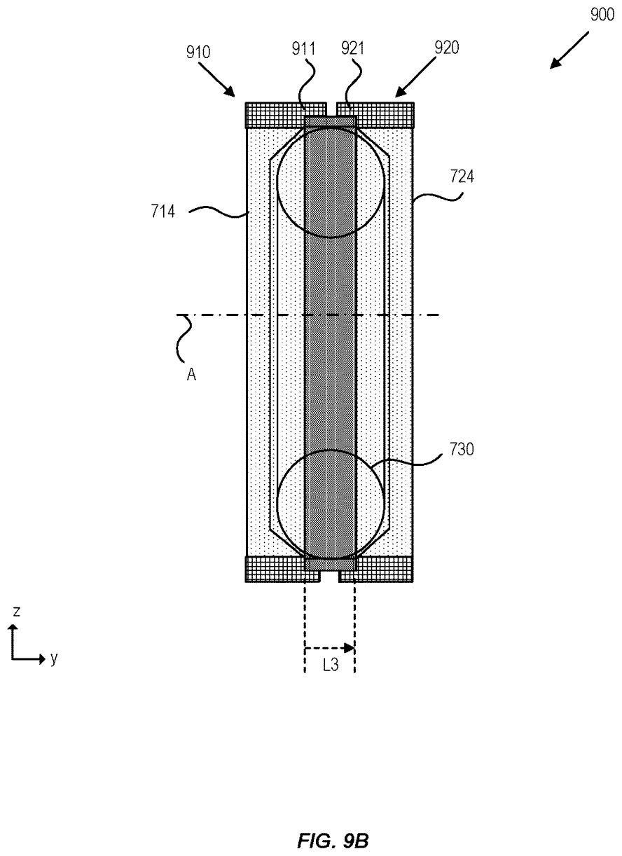

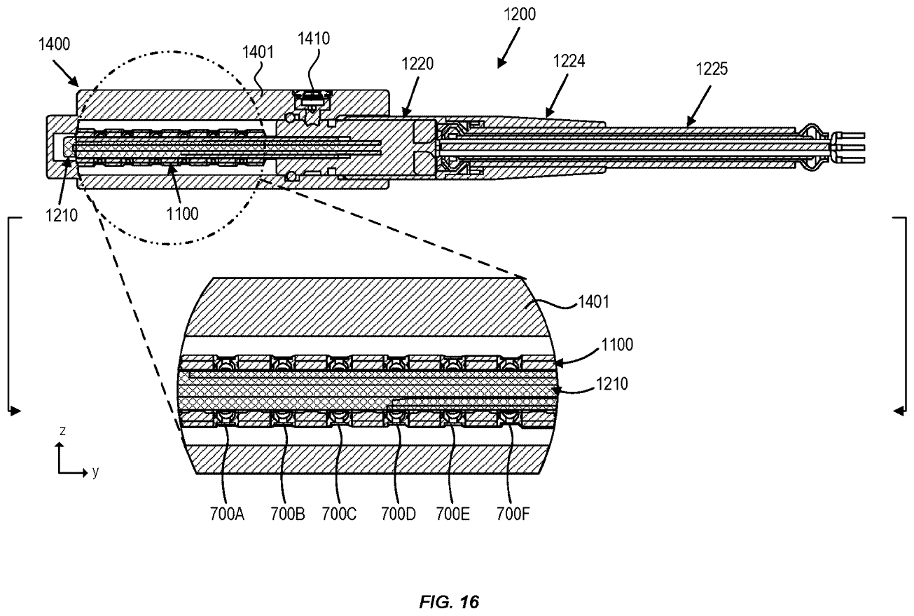

The circular coil spring 730 is retained between side surfaces of the first-side non-conductive retention member 710 and the second-side non-conductive retention member 720. In some embodiments, the retention members 710 and 720 may include a recessed portion 723 (see FIGS. 8 and 9A) within which a portion of the circular coil spring 730 can be retained. The circular coil spring 730 is annular in shape. Initially upon assembly, the circular coil spring 730 may not contact or only partially contact an inner surface 706 (see FIG. 8) of the annular conductive member 705. The circular coil spring 730 is configured expand and electrically couple with the annular conductive member 705 in response to a radially outward force. For example, an outer surface of the circular coil spring 730 directly contacts the inner surface 706 (see FIG. 8) of the annular conductive member 705 when the circular coil spring 730 is radially expanded (e.g., by inserting a male electrically connector in FIG. 16). The circular coil spring 730 can contract or retain its initial state when the radially outward force is removed. It can be understood that the circular coil spring is provided as an example of an element used for establishing electrical connection between male and female electrical connectors. The circular coil spring may be replaced with a canted coil spring or other donut shaped elements configured to expand or contract for establishing electrical connections. The circular springs can be helical springs that are in the shape of a circle or a donut. Canted springs are circular springs where the pitch of the spring is at a specific slant or canted angle.

In many embodiments, each of the first-side non-conductive retention member 710 and the second-side non-conductive retention member 720 have hollow cylindrical shape. The non-conductive members 710, 720 are coaxially coupled to the annular conductive member 705 along the central axis A. Each of the non-conductive retention members 710, 720 include a flange portion configured to interface with the annular end surfaces 708, 709, respectively, of the annular conductive member 705. In many embodiments, the non-conductive retention members 710, 720 have a stepped cylindrical shape including a first diameter portion configured to support the annular conductive member 705, and a flange portion extending from the first diameter portion. For example, the annular flange portion is a second diameter portion having a larger diameter than the first diameter portion. The first diameter portions face each other to support the annular conductive member 705. The second diameter portions can provide shoulders against which the end surfaces of the annular conductive member 705 can be retained to prevent axial movement.

For example, as illustrated in FIGS. 8 and 9, the first non-conductive retention member 710 has a first diameter portion 711 of diameter d1 and axial length L1 (see FIG. 9), and a second diameter portion 712 having a larger diameter d2 than the first diameter portion 711. The second diameter portion 712 corresponds to the annular flange portion. The second diameter portion 712 provides a shoulder against which the annular conductive member 705 can abut and restrict axial movement. Similarly, the second non-conductive retention member 720 has a first diameter portion 721 and a second diameter portion 722 having a larger diameter than the first diameter portion 721. The first diameter portions 711, 721 face each other to support the annular conductive member 705 and the second diameter portions 712, 722 provide shoulders to restrict axial movement of the annular conductive member 705. Accordingly, the annular conductive member 705 is securely retained over the first diameter portions 711, 721 of the non-conductive retention members 710, 720 such that the annular conductive member 705 is internally supported, as shown in FIG. 9A. For example, in FIG. 9A, outer cylindrical surfaces of the first diameter portions 711, 721 directly contact with the inner surface 706 of the annular conductive member 705. In some embodiments, the annular conductive member 705 has a cylindrical shape having an axial length L2. In some embodiments, the annular conductive member 705 may have a ring shape with a textured inner surface for securely engaging the first side and second side non-conductive retention members.

The first diameter portions 711, 721 each has the axial length L1 less than an axial length L2 of the annular conductive member 705. A total axial length of the first diameter portions 711, 721 of the non-conductive retention members 710, 720 is less than that of the annular conductive member 705 so that the circular coil spring 730 can be retained between side surfaces of the first portions 711, 721 of the first-side and the second-side non-conductive retention members 710, 720.

FIG. 9B illustrates a variation of a female contact assembly 900, where non-conductive retention members 910 and 920 may be configured to retain the annular conductive member 705 externally. For example, a cylindrical inner surface of the first-side non-conductive retention members 910, 920 is interfaced with end portions of the cylindrical outer surface 707 of the annular conductive member 705. For example, the non-conductive members 910, 920 may include internally stepped portions 911, 921, respectively, having inner surfaces extending over and interfacing with the outer surface 707 of the annular conductive member 705 and the internal steps serving as shoulders interfacing with end surfaces 708, 708 of the annular conductive member 705. The non-conductive retention members 910 and 920 can facilitate use of shorter length of the conductive member 705. For example, the conductive member 705 may have a length L3 shorter than length L2 (in FIG. 9A).

Typically electrical contacts of implantable devices have complex geometries and manufactured using expensive metal alloys that are not only electrically conductive but also biocompatible. These metal alloys are highly expensive and cost prohibitive when manufacturing complex geometries of an electrical contact. For example, typically an entire contact used in an implantable device is manufactured from the Pt/Ir alloy and has complex geometric shape necessitated by compact design specifications while providing high level of functionality for medical purposes. Manufacturing such contacts with complex geometries require several machine operations and material removal, which leads to wastage of expensive alloy material. Advantageously, the non-conductive retention members 710, 720 and a simple annular construction of the conductive member 705 of the present disclosure enables cost-effective manufacturing of implantable electrical connectors. As such, the present disclosure can facilitate cost-effective manufacturing of implantable electrical connectors of small (e.g., an internal diameter greater than 3.2 mm) as well as larger size (e.g., an internal diameter greater than 3.2 mm). For example, each of the first-side non-conductive retention member 710 and the second-side non-conductive retention member 720 can be made of polyetheretherketone (PEEK) or thermoplastic polyurethanes (TPU) or other moldable non-conductive polymer and/or ceramic materials, which can take complex geometric shapes and less expensive to manufacture. The annular conductive member 705 can be made of a conductive and corrosion resistant material such as platinum-iridium (Pt/Ir) alloy or other metals/metal alloys like MP35N, which will be less expensive to manufacture due to its simple and scalable geometry. The annular conductive member 705 can be extruded to form a cylindrical shape using the Pt/Ir material, for example. Extruding eliminates the loss of precious metal compared to than machining and making platinum chips. Furthermore, the female contact assembly 700 also provides modular scalability (e.g., diameter size and number of components that can be assembled) of the female electrical connector without adding exponential cost. Also, advantageously, existing or legacy implantable female electrical connector (e.g., within a header of the implantable device) can be retrofitted cost effectively.

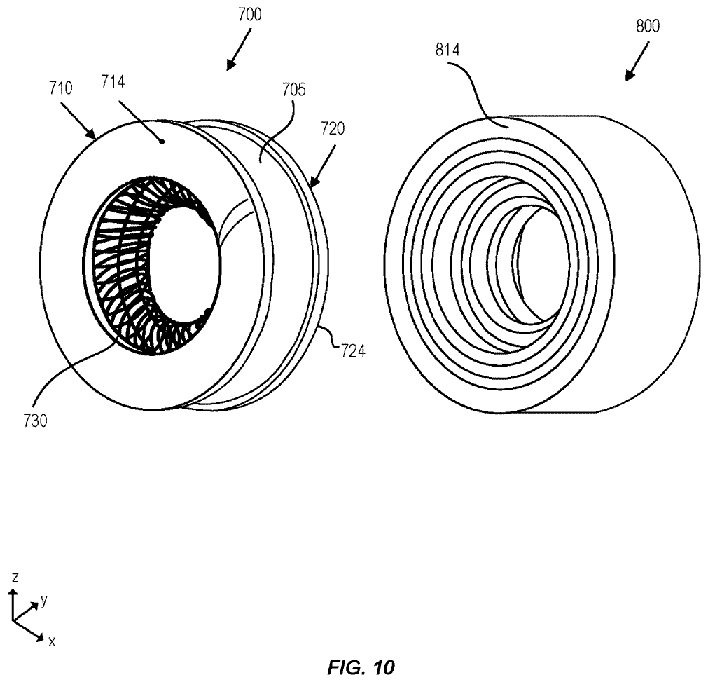

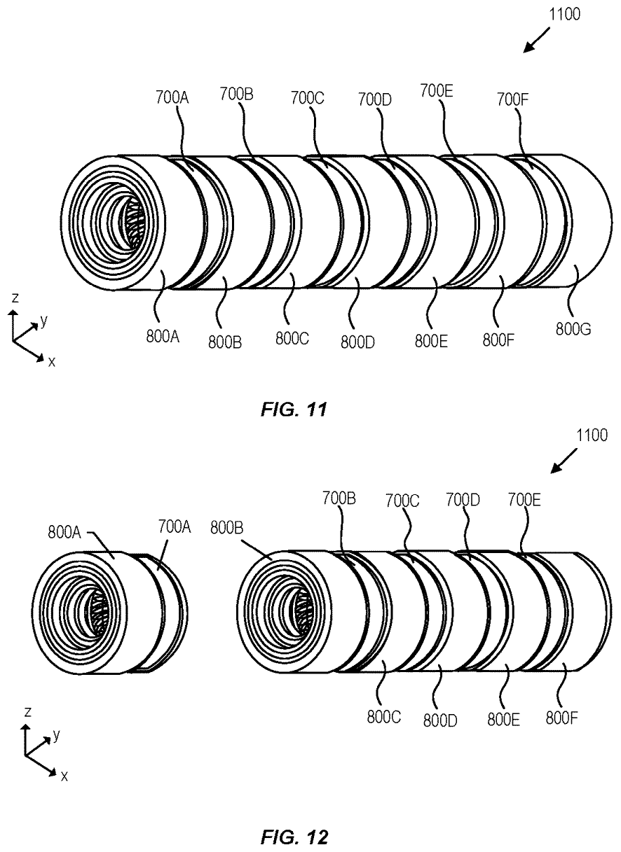

In many embodiments, the female electrical connector further includes one or more wiper seals and the female contact assembly 700 can be disposed between two wiper seals. For example, the female contact assembly 700 is configured to couple to a wiper seal 800, as shown in FIG. 10 through FIG. 12 to build female contact assemblies 1100, alternatively referred as a female contact stack 1100 hereafter for better readability. In the illustrated embodiment, the second diameter portions 712, 722 of the non-conductive retention members 710, 720 include end surfaces 714, 724 (see FIG. 8-10) configured to seal and couple with an end surface 814 of a wiper seal 800 (see FIG. 10).

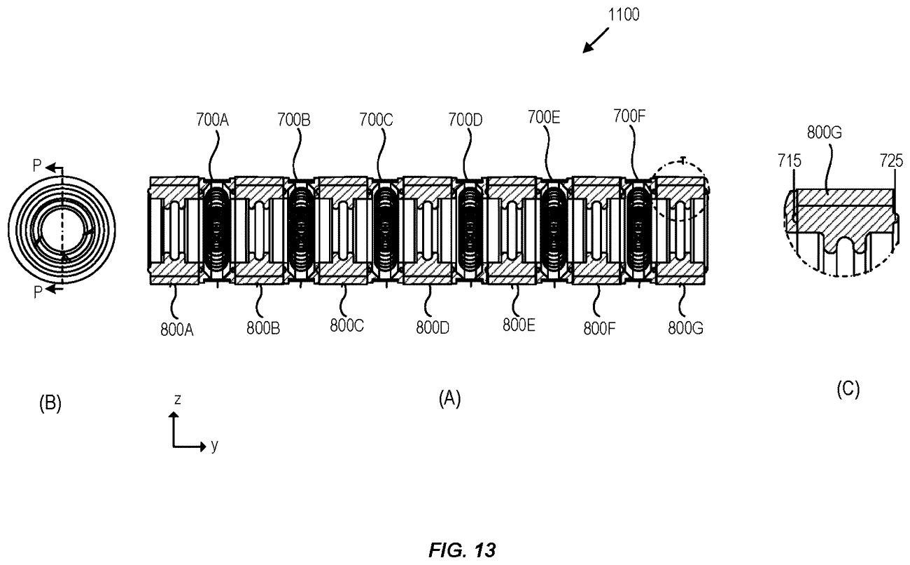

In an illustrated embodiment, as shown in FIG. 11 through FIG. 13, the female contact stack 1100 is formed by coupling a plurality of the female contact assembly 700 and a plurality of the wiper seals 800. FIG. 13 illustrates different views of the female contact stack 1100 including (A) a cross-section view, (B) a side view, and (C) an enlarged view of a portion of the female contact stack 1100. According to the illustrated example, the female contact stack 1100 include six individual female contact assembly coaxially stacked relative to each other. For example, the individual female contact assembly is identified as 700A, 700B, 700C, 700D, 700E, and 700F. In many embodiments, each individual female contact assembly is coupled to a wiper seal 800 to electrically isolate the female contact assemblies from each other. For example, an end surface (e.g., 714, 724) of a second diameter portion of a non-conductive retention member (e.g., 720) includes a sealing bump 715, 725 (see FIG. 13 (C)) configured to sealing engage a wiper seal (e.g., 800G) on either side.

In many embodiments, each of the female contact assemblies 700A-700F is disposed between two of the wiper seal assemblies (e.g., 800A and 800B). As shown, each wiper seal 800B-800G is disposed between adjacent female contact assemblies to isolate the female contact assemblies from each other. The wiper seals 800A and 800G are disposed at ends of the female contact stack 1100 and connected to corresponding female contact assembly 700A and 700F at these ends, respectively. The end wiper seals 800A and 800G further provides isolation and sealing from ingress of stray electrical currents or fluid from an external environment.

Furthermore, the first-side non-conductive retention members, the second-side non-conductive retention members and the annular conductive members of the female contact assemblies 700A-700F, and the wiper seals 800A-800G are coaxially stacked with respect to each other. The coaxial arrangement facilitates receiving of an elongated male electrical connector to establish electrical contact with each of the annular conductive members of the female contact assemblies 700A-700F.

FIG. 14 is an electrical connector including the female electrical connector 1400 and a male electrical connector 1200. The female electrical connector 1400 includes a connector body 1401 having an elongated receptacle 1403 configured to receive the female connector stack 1100 including the female contact assemblies 700A-700F. In many embodiments, the connector body 1401 may be formed (e.g., by over molding) over the female connector stack 1100. The female contact subassemblies 700A-700F are disposed in and distributed along the elongated receptacle 1403 for interfacing with the annular electrical contacts of the male electrical connector 1200.

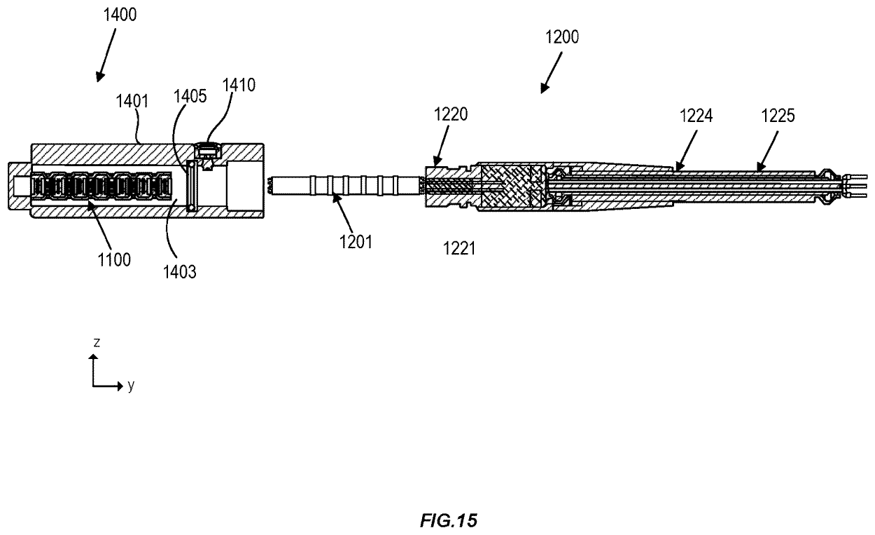

FIG. 15 and FIG. 16 show cross-section views of the electrical connector. In FIG. 15, the female electrical connector 1400 is uncoupled from the male electrical connector 1200. As illustrated, the connector body 1401 including the elongated receptacle 1403 receives the female contact stack 1100, which further receives the elongated electrical contact support member 1210 of the male electrical connector 1200. For example, in FIG. 16, the elongated electrical contact support member 1210 of the male electrical connector 1200 is inserted in the female electrical connector of 1400. The male electrical connector 1200 is retained to the connector body 1401 of the female electrical connector 1400 by a locking means 1410 (e.g., a set screw) provided at a proximal portion of the connector body 1401. For example, the locking means 1410 is a screw that can be adjusted (e.g., tightened or loosened) to engage or disengage the male electrical connector 1200. The elongated electrical contact support member 1210 is attached to a proximal lead housing 1220. The lead housing 1220 includes a slot or a groove 1221 configured to receive the locking means 1410, as shown in FIG. 15. The locking means 1410 such as the screw can be tightened to securely engage and prevent axial movement of the elongated electrical contact support member 1210 relative to the female contact stack 1100 once the elongated electrical contact support member 1210 is inserted therein.

The elongated electrical contact support member 1210 is an elongated shaft including a plurality of electrical contacts 1201-1206 axially spaced from each other. The relative location of the electrical contacts 1201-1206 correspond to locations of respective conductive members 705 of the plurality of female contact assemblies 700A-700F of the female contact stack 1100. Upon assembly, the electrical contacts 1201-1206 are electrically coupled to corresponding female contact assemblies 700A-700F. Each of the electrical contact of 1201-1206 can be connected to an electrical wire inside the elongated electrical contact support member 1210 and extend from within the elongated electrical contact support member 1210 to an electrical interface 1224 in the proximal lead housing 1220. The electrical interface 1224 include wires 1225 from the elongated electrical contact support member 1210 coupled to or grouped into or integrated within the implantable controller-to-VAD connection cable 26 (see FIG. 1) or the TETS power receiver-to-controller connection cable 28 to electrically couple the elongated electrical contact support member 1210 to VAD 14 or TETS power receiver 22 (see FIG. 1).

Upon inserting the elongated electrical contact support member 1210 into the female contact stack 1100, as shown in FIG. 16, the electrical contacts 1201-1206 are electrically coupled to corresponding respective conductive members 705 via the circular springs 730 of the female contact assemblies 700A-700F so that signals can be transmitted from the female contact assemblies 700A-700F to the male electrical contacts 1201-1206 and further to the electrical interface 1224. The electrical connection between connectors 700A-700F, 1201-1206, and 1224 facilitates power and communication transmission between the implantable controller and battery 20 via the female electrical connector assemblies 1100 within the connector body 1401 to the VAD 14, and/or TETS power receiver 22 (shown in FIG. 1). The connector assembly including the female contact stack 1100 coupled to the elongated electrical contact support member 1210 in the connector body 1401 can be electrically coupled with other electronic components of the implantable controller. For example, in FIG. 1, electronic components may be hermetically sealed in the housing 42 of the controller and battery 20. In some embodiments, feedthrough assemblies between the connector body 1401 (e.g., the header 41 in FIG. 1) and housing 42 of the controller and battery 20 facilitate electrical coupling of the connector assembly to the other electronic components of the controller and battery 20 (e.g., the processor 56 configured to control VAD speed, the memory 54 to store data, the rechargeable battery 52 to store power received from the TETS power receiver 22, etc., as shown in FIG. 6 above).

In many embodiments, the wiper seals 800A-800G provide electrical isolation between the contacts 700A-700F and 1201-1206 and sealing against fluid ingress therebetween. The non-conductive members 710, 720 also provide additional electrical isolation and sealing against fluid ingress. A wiper seal includes an annular housing and an annular seal supported by the annular housing and configured to block passage of fluid pas the annular seal. In many embodiments, the annular seal is configured to sealing engage the male electrical connector. For example, each of the wiper seals 800A-800G includes a tongue or a flap (e.g., wiper blade style) configured to isolate an electrical contact (e.g., 700A) from other electrical contacts (e.g., 700B). The wiper tongue or flap is a compliant member that allows for adequate contact isolation while facilitating low lead insertion force. Many high-powered medical implant applications, in addition to wiper seals include distal or proximal seals to isolate electrical contacts, particularly the electrical contacts at a proximal end and a distal end of the female contact stack 1100. For example, the wiper tongue on a proximal end and/or a distal end of the female contact stack 1100 may be insufficient for inhibiting stray electrical currents from entering the electrical contacts and/or inhibiting fluid ingress from the outside environment. To boost sealing performance and electrical isolation of the header, additional sealing elements 1405 are provided at the proximal end of the elongated receptacle 1403 (see FIGS. 14-15).

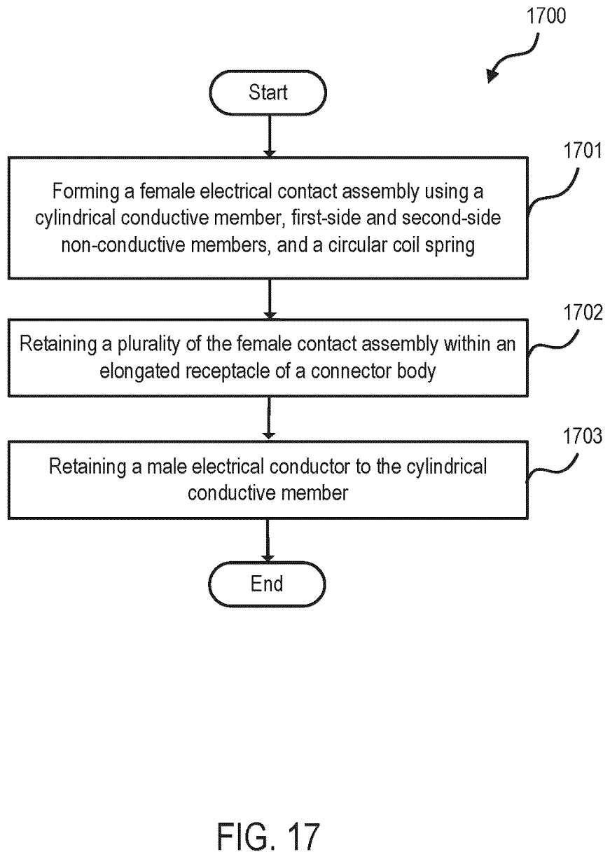

FIG. 17 is a method of fabricating an implantable female electrical connector assembly (e.g., 1400) for an implantable medical device. The method 1700 can be implemented as steps 1701, 1702, and 1703, according to an example. Step 1701 involves forming a female contact assembly (e.g., 700 in FIGS. 7 and 8) by retaining a circular coil spring (e.g., 730 in FIGS. 7 and 8) within a cylindrical conductive member (e.g., 705 in FIGS. 7 and 8) via a first-side non-conductive member (e.g., 710 in FIGS. 7 and 8) mounted to the cylindrical conductive member at a first end of the cylindrical conductive member and a second-side non-conductive member (e.g., 720 in FIGS. 7 and 8) mounted to the cylindrical conductive member at a second end of the cylindrical conductive member. Step 1702 involves retaining the plurality of female contact subassemblies (e.g., 1100 in FIG. 11) within an elongated receptacle of a connector body.

In many embodiments, the plurality of female contact assemblies (e.g., 700A-700F in FIG. 11) are coaxially stacked such that each female contact assembly is spaced from each other. The stacked female contact assemblies (e.g., 700A-700F in FIG. 11) is disposed within the elongated receptacle of the connector body (e.g., the receptacle 1403 of the connector body 1401 in FIG. 14). In many embodiments, a wiper seal (e.g., 800A-800G in FIG. 11) is disposed between adjacent female contact assemblies.

In many embodiments, the connector body (e.g., 1401) is retained to a housing of the implantable medical device. The connector body can be a header of an implantable medical device. For example, in FIG. 1, the connector body (e.g., 1401) may be the header 41 including two laterally spaced elongated receptacles (e.g., instances of 1403), each elongated receptacle including a female contact (e.g., 1100 of FIG. 11).

In many embodiments, the method 1700 further includes step 1703 that involves retaining a male electrical conductor (e.g., 1200 in FIG. 14) to the cylindrical conductive member. For example, the male electrical conductor (e.g., 1200) includes an elongated electrical contact support member (e.g., 1210) and annular electrical contacts (e.g., 1201-1206) mounted to and spaced apart along the elongated electrical contact support member (e.g., 1210).

Thus, advantageously, different components such as the non-conductive memebers 710, 720, the conductive member 705, the circular spring 730, and/or the wiper seal 800 can be manufactured sperately using cost-effective manufacturing processes and material and assembled together to provide a desired fuctionality of an implantable device. As such, both manufacturing and assembly resources can be effectively used making the implantable device affordable and timely available for large group of patients.

### Example Embodiments

Example 1 is an implantable electrical connector assembly that includes a male electrical connector and a female electrical connector. The male electrical connector includes an elongated electrical contact support member and annular electrical contacts mounted to and spaced apart along the elongated electrical contact support member. The female electrical connector includes a connector body having an elongated receptacle and female contact assemblies disposed in and distributed along the elongated receptacle for interfacing with the annular electrical contacts of the male electrical connector. Each of the female contact assemblies includes a circular coil spring, an annular conductive member, a first-side non-conductive retention member, and a second-side non-conductive retention member. The circular coil spring is disposed within the annular conductive member. The first-side non-conductive retention member is mounted to the annular conductive member and disposed on a first side of the circular coil spring. The second-side non-conductive retention member is mounted to the annular conductive member and disposed on a second side of the circular coil spring opposite to the first side of the circular coil spring. The circular coil spring is retained between and by the first-side non-conductive retention member and the second-side non-conductive retention member.

Example 2 is the implantable electrical connector of example 1, wherein the annular conductive member has a cylindrical shape.

Example 3 is the implantable electrical connector of example 2, wherein the annular conductive member is formed from a cylindrical extrusion.

Example 4 is the implantable electrical connector of example 2, wherein the annular conductive member includes a cylindrical inner surface, an annular first end surface, and an annular second end surface, a cylindrical outer surface of the first-side non-conductive retention member is interfaced with a first end portion of the cylindrical inner surface of the annular conductive member, an annular flange portion of the first-side non-conductive retention member is interfaced with the annular first end surface of the annular conductive member; a cylindrical outer surface of the second-side non-conductive retention member is interfaced with a second end portion of the cylindrical inner surface of the annular conductive member, an annular flange of the second-side non-conductive retention member is interfaced with the annular second end surface of the annular conductive member, and the circular coil spring is configured to be interfaced with a central portion of the annular conductive member that extends between the first end portion and the second end portion of the cylindrical inner surface to electrically couple the annular conductive member with a respective one of the annular electrical contacts of the male electrical connector.

Example 5 is the implantable electrical connector of example 4, wherein the female electrical connector further includes wiper seal assemblies, each of the female contact assemblies is disposed between two of the wiper seal assemblies, and each of the wiper seal assemblies includes an annular housing and an annular seal supported by the annular housing. The annular seal is configured to sealing engage the male electrical connector. The wiper seal assembly is configured to cooperate with the male electrical connector to block passage of fluid along the male electrical connector.

Example 6 is the implantable electrical connector of example 2, wherein the annular conductive member includes a cylindrical outer surface, an annular first end surface, and an annular second end surface, a cylindrical inner surface of the first-side non-conductive retention member is interfaced with a first end portion of the cylindrical outer surface of the annular conductive member, an annular flange of the first-side non-conductive retention member is interfaced with the annular first end surface of the annular conductive member, a cylindrical inner surface of the second-side non-conductive retention member is interfaced with a second end portion of the cylindrical outer surface of the annular conductive member, an annular flange of the second-side non-conductive retention member is interfaced with the annular second end surface of the annular conductive member, and the circular coil spring is configured to be interfaced with a central portion of the annular conductive member that extends between the first end portion and the second end portion of the cylindrical inner surface to electrically couple the annular conductive member with a respective one of the annular electrical contacts of the male electrical connector.

Example 7 is the implantable electrical connector of example 6, wherein the female electrical connector further includes wiper seal assemblies, each of the female contact assemblies is disposed between two of the wiper seal assemblies, and each of the wiper seal assemblies includes an annular housing and an annular seal supported by the annular housing. The annular seal is configured to sealing engage the male electrical connector. The wiper seal assembly is configured to cooperate with the male electrical connector to block passage of fluid along the male electrical connector.

Example 8 is the implantable electrical connector of any one of examples 2 through 7, wherein the annular conductive member has an internal diameter greater than 3.2 mm.

Example 9 is the implantable electrical connector of any one of examples 1 through 7, wherein each of the first-side non-conductive retention member and the second-side non-conductive retention member is made of polyetheretherketone (PEEK) or thermoplastic polyurethanes (TPU).

Example 10 is the implantable electrical connector of any one of examples 1 through 7, wherein the annular conductive member is made of a platinum-iridium alloy.

Example 11 is a method of fabricating a female electrical connector of an implantable electrical connector assembly. The method includes receiving a connector body that includes an elongated receptacle, receiving female contact assemblies, and retaining the female contact assemblies within the elongated receptacle of the connector body. Each of the female contact assemblies includes a cylindrical conductive member, a circular coil spring, a first-side non-conductive retention member, and a second-side non-conductive retention member. The circular coil spring is disposed within the cylindrical conductive member. The first-side non-conductive retention member is mounted to the cylindrical conductive member and disposed on a first side of the circular coil spring. The second-side non-conductive retention member is mounted to the cylindrical conductive member and disposed on a second side of the circular coil spring opposite to the first side of the circular coil spring. The circular coil spring is retained between and by the first-side non-conductive retention member and the second-side non-conductive retention member.

Example 12 is the method of example 11, further including retaining wiper seal assemblies within elongated receptacle of the connector body.

Example 13 is the method of example 12, wherein each of the female contact assemblies is disposed between two of the wiper seal assemblies.

Example 14 is the method of example 13, wherein each of the wiper seal assemblies includes an annular housing and an annular seal supported by the annular housing and is configured to sealing engage a male electrical connector and each of the wiper seal assemblies configured to cooperate with the male electrical connector to block passage of fluid.

Example 15 is the method of example 11, wherein the cylindrical conductive member includes a cylindrical inner surface, an annular first end surface, and an annular second end surface, a cylindrical outer surface of the first-side non-conductive retention member is interfaced with a first end portion of the cylindrical inner surface of the cylindrical conductive member, an annular flange portion of the first-side non-conductive retention member is interfaced with the annular first end surface of the cylindrical conductive member, a cylindrical outer surface of the second-side non-conductive retention member is interfaced with a second end portion of the cylindrical inner surface of the cylindrical conductive member, an annular flange of the second-side non-conductive retention member is interfaced with the annular second end surface of the cylindrical conductive member, and the circular coil spring is configured to be interfaced with a central portion of the cylindrical conductive member that extends between the first end portion and the second end portion of the cylindrical inner surface to electrically couple the cylindrical conductive member with a respective one of annular electrical contacts of a male electrical connector.

Example 16 is the method of example 11, wherein the cylindrical conductive member includes a cylindrical outer surface, an annular first end surface, and an annular second end surface, a cylindrical inner surface of the first-side non-conductive retention member is interfaced with a first end portion of the cylindrical outer surface of the cylindrical conductive member, an annular flange of the first-side non-conductive retention member is interfaced with the annular first end surface of the cylindrical conductive member, a cylindrical inner surface of the second-side non-conductive retention member is interfaced with a second end portion of the cylindrical outer surface of the cylindrical conductive member, an annular flange of the second-side non-conductive retention member is interfaced with the annular second end surface of the cylindrical conductive member, and the circular coil spring is configured to be interfaced with a central portion of the cylindrical conductive member that extends between the first end portion and the second end portion of the cylindrical inner surface to electrically couple the cylindrical conductive member with a respective one of annular electrical contacts of a male electrical connector.

Example 17 is the method of any one of examples 11 through 16, further including supporting the connector body via a housing of an implantable medical device.

Example 18 is the method of any one of examples 11 through 16, wherein each of the first-side non-conductive retention member and the second-side non-conductive retention member and second-side non-conductive members is made of polyetheretherketone (PEEK) or thermoplastic polyurethanes (TPU).

Example 19 is the method of any one of examples 11 through 16, wherein the cylindrical conductive member is made of a platinum-iridium alloy.

Example 20 is the method of any one of examples 11 through 16, wherein the cylindrical conductive member has an internal diameter greater than 3.2 mm.

It is to be understood that terms such as “distal,” “proximal,” “side,” “inner,” and the like that can be used herein merely describe points of reference and do not necessarily limit embodiments of the present disclosure to any particular orientation and/or configuration. As used herein, “proximal” refers to a direction toward the end of the female contact stack near the clinician and “distal” refers to a direction away from the clinician and (generally) inside the body of a patient. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

The terms “longitudinal,” “axial” or “axially” are generally longitudinal as used herein to describe the relative position related to a female contact stack or other components of the system herein. For example, “longitudinal” or “axial” indicates an axis passing along a center of a female contact stack from a proximal end to a distal end. The term “radial” generally refers to a direction perpendicular to the “axial” direction.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions, and changes in the form of the methods, apparatuses and systems described herein can be made without departing from the spirit of the present disclosures. The accompanying claims and their equivalents are intended to cover such forms or modifications as would fall within the scope and spirit of the present disclosures.

What is claimed is:

1. An implantable electrical connector assembly comprising:
a male electrical connector comprising an elongated electrical contact support member and annular electrical contacts mounted to and spaced apart along the elongated electrical contact support member; and
a female electrical connector comprising a connector body having an elongated receptacle and female contact assemblies disposed in and distributed along the elongated receptacle for interfacing with the annular electrical contacts of the male electrical connector, wherein each of the female contact assemblies comprises a circular coil spring, an annular conductive member, a first-side non-conductive retention member, and a second-side non-conductive retention member, wherein the circular coil spring is disposed within the annular conductive member, wherein the first-side non-conductive retention member is mounted to the annular conductive member and disposed on a first side of the circular coil spring, wherein the second-side non-conductive retention member is mounted to the annular conductive member and disposed on a second side of the circular coil spring opposite to the first side of the circular coil spring, and wherein the circular coil spring is retained between and by the first-side non-conductive retention member and the second-side non-conductive retention member.

2. The implantable electrical connector assembly of claim 1, wherein the annular conductive member has a cylindrical shape.

3. The implantable electrical connector assembly of claim 2, wherein the annular conductive member is formed from a cylindrical extrusion.

4. The implantable electrical connector assembly of claim 2, wherein:
the annular conductive member comprises a cylindrical inner surface, an annular first end surface, and an annular second end surface;
a cylindrical outer surface of the first-side non-conductive retention member is interfaced with a first end portion of the cylindrical inner surface of the annular conductive member;
an annular flange portion of the first-side non-conductive retention member is interfaced with the annular first end surface of the annular conductive member;
a cylindrical outer surface of the second-side non-conductive retention member is interfaced with a second end portion of the cylindrical inner surface of the annular conductive member;
an annular flange of the second-side non-conductive retention member is interfaced with the annular second end surface of the annular conductive member; and
the circular coil spring is configured to be interfaced with a central portion of the annular conductive member that extends between the first end portion and the second end portion of the cylindrical inner surface to electrically couple the annular conductive member with a respective one of the annular electrical contacts of the male electrical connector.

5. The implantable electrical connector assembly of claim 4, wherein:
the female electrical connector further comprises wiper seal assemblies;
each of the female contact assemblies is disposed between two of the wiper seal assemblies; and
each of the wiper seal assemblies comprises an annular housing and an annular seal supported by the annular housing, wherein the annular seal is configured to sealing engage the male electrical connector, and the wiper seal assembly is configured to cooperate with the male electrical connector to block passage of fluid along the male electrical connector.

6. The implantable electrical connector assembly of claim 2, wherein:
the annular conductive member comprises a cylindrical outer surface, an annular first end surface, and an annular second end surface;
a cylindrical inner surface of the first-side non-conductive retention member is interfaced with a first end portion of the cylindrical outer surface of the annular conductive member;
an annular flange of the first-side non-conductive retention member is interfaced with the annular first end surface of the annular conductive member;
a cylindrical inner surface of the second-side non-conductive retention member is interfaced with a second end portion of the cylindrical outer surface of the annular conductive member;
an annular flange of the second-side non-conductive retention member is interfaced with the annular second end surface of the annular conductive member; and
the circular coil spring is configured to be interfaced with a central portion of the annular conductive member that extends between the first end portion and the second end portion of the cylindrical inner surface to electrically couple the annular conductive member with a respective one of the annular electrical contacts of the male electrical connector.

7. The implantable electrical connector assembly of claim 6, wherein:
the female electrical connector further comprises wiper seal assemblies;
each of the female contact assemblies is disposed between two of the wiper seal assemblies; and
each of the wiper seal assemblies comprises an annular housing and an annular seal supported by the annular housing, wherein the annular seal is configured to sealing engage the male electrical connector, and the wiper seal assembly is configured to cooperate with the male electrical connector to block passage of fluid along the male electrical connector.

8. The implantable electrical connector assembly of claim 2, wherein the annular conductive member has an internal diameter greater than 3.2 mm.

9. The implantable electrical connector assembly of claim 1, wherein each of the first-side non-conductive retention member and the second-side non-conductive retention member is made of polyetheretherketone (PEEK) or thermoplastic polyurethanes (TPU).

10. The implantable electrical connector assembly of claim 1, wherein the annular conductive member is made of a platinum-iridium alloy.

11. A method of fabricating a female electrical connector of an implantable electrical connector assembly, the method comprising:
receiving a connector body comprising an elongated receptacle;
receiving female contact assemblies, wherein each of the female contact assemblies comprises a cylindrical conductive member, a circular coil spring, a first-side non-conductive retention member, and a second-side non-conductive retention member, wherein the circular coil spring is disposed within the cylindrical conductive member, wherein the first-side non-conductive retention member is mounted to the cylindrical conductive member and disposed on a first side of the circular coil spring, wherein the second-side non-conductive retention member is mounted to the cylindrical conductive member and disposed on a second side of the circular coil spring opposite to the first side of the circular coil spring, and wherein the circular coil spring is retained between and by the first-side non-conductive retention member and the second-side non-conductive retention member; and
retaining the female contact assemblies within the elongated receptacle of the connector body.

12. The method of claim 11, further comprising retaining wiper seal assemblies within elongated receptacle of the connector body.

13. The method of claim 12, wherein each of the female contact assemblies is disposed between two of the wiper seal assemblies.

14. The method of claim 13, wherein:
each of the wiper seal assemblies comprises an annular housing and an annular seal supported by the annular housing and configured to sealing engage a male electrical connector; and
each of the wiper seal assemblies is configured to cooperate with the male electrical connector to block passage of fluid.

15. The method of claim 11, wherein:
the cylindrical conductive member comprises a cylindrical inner surface, an annular first end surface, and an annular second end surface;
a cylindrical outer surface of the first-side non-conductive retention member is interfaced with a first end portion of the cylindrical inner surface of the cylindrical conductive member;
an annular flange portion of the first-side non-conductive retention member is interfaced with the annular first end surface of the cylindrical conductive member;
a cylindrical outer surface of the second-side non-conductive retention member is interfaced with a second end portion of the cylindrical inner surface of the cylindrical conductive member;
an annular flange of the second-side non-conductive retention member is interfaced with the annular second end surface of the cylindrical conductive member; and
the circular coil spring is configured to be interfaced with a central portion of the cylindrical conductive member that extends between the first end portion and the second end portion of the cylindrical inner surface to electrically couple the cylindrical conductive member with a respective one of annular electrical contacts of a male electrical connector.

16. The method of claim 11, wherein:
the cylindrical conductive member comprises a cylindrical outer surface, an annular first end surface, and an annular second end surface;
a cylindrical inner surface of the first-side non-conductive retention member is interfaced with a first end portion of the cylindrical outer surface of the cylindrical conductive member;
an annular flange of the first-side non-conductive retention member is interfaced with the annular first end surface of the cylindrical conductive member;
a cylindrical inner surface of the second-side non-conductive retention member is interfaced with a second end portion of the cylindrical outer surface of the cylindrical conductive member;
an annular flange of the second-side non-conductive retention member is interfaced with the annular second end surface of the cylindrical conductive member; and
the circular coil spring is configured to be interfaced with a central portion of the cylindrical conductive member that extends between the first end portion and the second end portion of the cylindrical inner surface to electrically couple the cylindrical conductive member with a respective one of annular electrical contacts of a male electrical connector.

17. The method of claim 11, further comprising supporting the connector body via a housing of an implantable medical device.

18. The method of claim 11, wherein each of the first-side non-conductive retention member and the second-side non-conductive retention member and second-side non-conductive members is made of polyetheretherketone (PEEK) or thermoplastic polyurethanes (TPU).

19. The method of claim 11, wherein the cylindrical conductive member is made of a platinum-iridium alloy.

20. The method of claim 11, wherein the cylindrical conductive member has an internal diameter greater than 3.2 mm.
